(19) United States
(12) Patent Application Publication
(10) Pub. No.: US 2018/0338719 A1
(43) Pub. Date: Nov. 29, 2018

(54) BIOLOGICAL SENSOR FOR OBTAINING INFORMATION ON LIVING BODY

(71) Applicant: Panasonic Intellectual Property Management Co., Ltd., Osaka (JP)

(72) Inventors: YOSHIHIRO TOMITA, Osaka (JP); KOICHI HIRANO, Osaka (JP); SUSUMU SAWADA, Osaka (JP); HIDEKI OHMAE, Hyogo (JP)

(21) Appl. No.: 15/961,388

(22) Filed: Apr. 24, 2018

(30) Foreign Application Priority Data
May 26, 2017 (JP) 2017-105097

(51) Int. Cl. A61B 5/00; A61B 5/04

(52) U.S. Cl. CPC A61B 5/6801; A61B 5/04085; A61B 5/04

(57) ABSTRACT

A biological sensor includes: a first sheet that has flexibility and/or stretchability; and a second sheet that has flexibility and/or stretchability. The second sheet has a first surface facing the first sheet, and a second surface opposite to the first surface. The second surface has a plurality of projections that are configured to be brought into contact with a living body to obtain information on the living body. In the second surface, at least part of each of the plurality of projections has conductivity. In the first surface, a portion surrounding each of the plurality of projections is bonded to the first sheet. Within each of the plurality of projections, an enclosed space defined by the first surface of the second sheet and the first sheet is present

### BACKGROUND

### 1. Technical Field

The present disclosure relates to a biological sensor that obtains information on a living body.

### 2. Description of the Related Art

In recent years, biological sensors which obtain vital signs information on a living body, such as electrocardiogram, brain waves, and electromyogram, have been used in a medical field to diagnose illness or health conditions.

For instance, U.S. Pat. No. 4,419,998 discloses a biological electrode system using a disposable electrode set. Also, Japanese Unexamined Patent Application Publication No. 63-024928 discloses a medical electrode that uses hydrophilic gel as a skin interface conductive member. Also, Japanese Unexamined Patent Application Publication (Translation of PCT Application) No. 7-503628 discloses a structure that relates to a biological potential electrode and that maintains a depolarization state of an electrode before use.

These are formulated as medical devices in medical systems, and cannot be used for monitoring in daily life.

However, in recent years, biological sensors have been advanced as wearable devices. Thus, even during a daily activity, vital signs information on a body can be obtained via attachment of a biological sensor to the body.

For instance, Japanese Unexamined Patent Application Publication No. 2016-158912 discloses clothes for monitoring biological signals, having an electrode for detecting biological signals. Also, Japanese Unexamined Patent Application Publication No. 2014-151018 discloses a conductive textile that allows an electrode or a wire in any shape to be formed with high accuracy. The conductive textile is wearable as tailored clothes, and is useful as a light-weighted, thin wearable biological electrode that has excellent wearing performance. Also, Japanese Unexamined Patent Application Publication No. 2016-112384 discloses a smart biological detection clothes for measuring electrocardiogram.

### SUMMARY

One non-limiting and exemplary embodiment provides a biological sensor that gives an excellent sense of wearing and can stably detect weak biological signals.

In one general aspect, the techniques disclosed here feature a biological sensor including: a first sheet that has flexibility and/or stretchability; and a second sheet that has flexibility and/or stretchability. The second sheet has a first surface facing the first sheet, and a second surface opposite to the first surface. The second surface has a plurality of projections that are configured to be brought into contact with a living body to obtain information on the living body. In the second surface, at least part of each of the plurality of projections has conductivity. In the first surface, a portion surrounding each of the plurality of projections is bonded to the first sheet. Within each of the plurality of projections, an enclosed space defined by the first surface of the second sheet and the first sheet is present.

It should be noted that general or specific embodiments may be implemented as a sensor, a device, a system, a method, an integrated circuit, a computer program, a storage medium, or any selective combination thereof.

A biological sensor according to an aspect of the present disclosure provides an excellent sense of wearing and can stably detect weak biological signals.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a sectional perspective view schematically illustrating a biological sensor according to a first embodiment;

FIG. 2 is a diagram illustrating an example of use of the biological sensor according to the first embodiment;

FIG. 3 is a sectional view schematically illustrating the biological sensor when a user wears clothes equipped with the biological sensor in the example of use of FIG. 2;

FIG. 4 is a sectional view schematically illustrating the biological sensor when a user wears clothes equipped with the biological sensor and performs an activity in the example of use of FIG. 2;

FIG. 5 is a diagram explaining the biological sensor and the state of a living body surface while a user is performing an activity in the example of use of FIG. 2;

FIG. 6 is a sectional perspective view schematically illustrating a biological sensor according to a modification of the first embodiment;

FIG. 7 is a sectional perspective view schematically illustrating the biological sensor according to a second embodiment;

FIG. 8 is a sectional view schematically illustrating the manner in which the biological sensor according to the second embodiment is in intimate contact with a living body surface;

FIG. 9 is a sectional perspective view schematically illustrating a biological sensor according to a first modification of the second embodiment; and

FIG. 10 is a diagram explaining a method of manufacturing a biological sensor according to a second modification of the second embodiment.

### DETAILED DESCRIPTION

### Underlying Knowledge Forming Basis of the Present Disclosure

First, the underlying knowledge forming the basis of a biological sensor in the present disclosure devised by the inventors will be described.

Biological sensors in related art are principally used as measuring devices that measure bioelectric potentials, such as cardiac potentials, muscle potentials, and brain waves. For instance, a biological sensor as a medical device is for the purpose of diagnosing illness or health conditions in a medical field, such as a hospital, and a non-portable device is used. Also, as disclosed in Japanese Unexamined Patent Application Publication No. 63-024928, so-called “a gel electrode” having a pad shape, in which hydrophilic gel is immersed, is used as a biological electrode for obtaining bioelectric potentials. The hydrophilic gel has conductivity and viscosity. Thus, the gel electrode can stably maintain an electrical connection to the surface of a living body (U.S. Pat. No. 4,419,998, Japanese Unexamined Patent Application Publication No. 63-024928, and Japanese Unexamined Patent Application Publication (Translation of PCT Application) No. 7-503628).

In recent years, due to the emergence of wearable devices that can obtain vital signs information from a living body, not only in a medical field, but also in daily life, vital signs information can be obtained from a living body using a biological sensor more easily. In conjunction with this, the need has increased for utilization of vital signs information in services, such as measurement of amount of activity in daily health care, fitness, or sports. Thus, biological sensors that can measure bioelectric potentials, such as cardiac potentials, muscle potentials, and brain waves more easily are demanded (Japanese Unexamined Patent Application Publication Nos. 2016-158912, 2014-151018, and 2016-112384).

For instance, examples of a biological sensor which can measure heart rate more easily include myBeat (registered trademark) manufactured by UNION TOOL CO. and hitoe (registered trademark) manufactured by NTT DoCoMo Inc.

The myBeat (registered trademark) is a biological sensor in which a compact signal processing circuit unit is connected to a disposable electrode, and which converts obtained biological signals to electrical signals, and can wirelessly transmit the electrical signals to an external terminal such as a smartphone. Thus, even when a user performs an activity such as daily life and exercise with the electrode attached to the chest, myBeat (registered trademark) can measure cardiac potentials.

Also, the hitoe (registered trademark) is a biological sensor in which a compact signal processing circuit unit is connected to a biological electrode using conductive fibers provided inside a sportswear, and which converts obtained biological signals to electrical signals, and can wirelessly transmit the electrical signals to an external terminal such as a smartphone. Thus, the hitoe (registered trademark) can easily measure cardiac potentials by only being worn.

These biological sensors can analyze an electrocardiogram waveform with an external terminal by wirelessly transmitting obtained cardiac potentials to the external terminal. Therefore, in daily life, a user not only can easily obtain the vital signs information on himself or herself, but also can share information with a doctor or a family. Thus, it is expected to use biological sensors for more accurate treatment, prevention, or health management.

Here, the form of electrode of biological sensor is broadly classified into two types. One of the types is a wet electrode, such as a gel electrode and a disposable electrode, in which conductive liquid gel, that is, wet gel or a wet conductive material having adhesiveness is used. The other type is a dry electrode in which clothing is tailored with conductive fibers and conductive gel is not used.

The wet electrode can be directly attached to the surface of a living body, and can reliably establish electrical connection to the surface of a living body by conductive wet gel or the like. Thus, stable biological sensing is possible. On the other hand, since gel or adhesive of the wet electrode is directly brought into contact with the surface of a living body, uncomfortable touch, such as slimy or cool touch is caused at the time of attachment or detachment of the wet electrode. Also, in order to attach the wet electrode to the surface a living body for a long time, it is necessary to consider the effect of the wet electrode on the skin. In a medical field, weak biological signals have to be measured with high sensitivity. Thus, a higher priority is placed on the sensitivity of a biological sensor rather than the sense of wearing and the effect of the sensor on the skin. However, when a general user wears a biological sensor on a daily basis, the sense of wearing and the effect of the sensor on the skin is more important than the sensitivity of the sensor. Also, when a user performs an activity causing much perspiration, such as sports with a wet electrode attached to the living body surface, a large quantity of sweat accumulates between the electrode and the living body surface or the gel contained in the electrode flows out by the sweat, and the sense of wearing of the biological sensor is further worsened. Also, the skin may be effected by the sweat accumulated between the electrode and the living body surface. When the quantity of sweat is further increased, a layer of sweat is formed between the electrode and the living body surface, and the electrode may fall off from the living body surface.

In contrast, in a dry electrode using conductive fibers or the like, the effect on the skin is improved. However, a problem arises in that it is difficult to reliably ensure electrical connection between the electrode and the living body surface by bringing the dry electrode into intimate contact with the living body surface as in the case of a wet electrode. Therefore, in a sportswear biological sensor represented by the hitoe (registered trademark), particularly portions of both chests, in which dry electrodes are disposed, are fastened using clothes having a large restraining force, called compression wear, and adhesiveness between the dry electrode and the living body surface is improved. However, when a fastening pressure of the wear applied to the living body surface is increased to improve the adhesiveness between the electrode and the living body surface, uncomfortable sense such as sense of restraint or sense of strangeness is brought to a user. Thus, the fastening force has a certain limit. Even if the living body surface is tightly fastened by the clothes, the material of the clothes slides relative to the living body surface due to body motion at the time of sports activity or the like. In a wearable biological sensor, the electrode and the clothes are integrated. Thus, the entire clothes move, following expansion and contraction of a portion of the living body surface, in which body motion is vigorous. Therefore, a wearable biological sensor tends to be affected by a positional displacement between the living body surface and the electrode, and poor adhesion, as compared with the case of a biological sensor using a wet electrode. This causes noise of biological sensing by a biological sensor using a dry electrode.

As described above, a wet electrode has a problem, such as sense of wearing and an effect on the skin, and a problem arises in that it is difficult to stably ensure electrical connection between the dry electrode and the living body surface. Also, the dry electrode has a problem in that even when a fastening pressure of the clothes applied to the living body surface is increased to improve the adhesiveness between the electrode and the living body surface, uncomfortable sense such as sense of restraint is brought to a user. Thus, sense of wearing is worsened.

The inventors have intensively studied to cope with the above-mentioned problem. As a result, the inventors have found that when multiple projections each having an elastic structure are provided with predetermined intervals on one surface of an expandable and contractible base material of a biological sensor, even in a dry electrode for which gel is not used, elastic projections conform to and come into intimate contact with the living body surface. In addition, the inventors have found that the surfaces of multiple projections of a biological sensor are each provided with a conductive pattern. Thus, even when a pressure (hereinafter, referred to as a restraining pressure) that restrains the living body surface to the electrode is low, electrical connection between the living body surface and the electrode is stably ensured.

The present disclosure provides a biological sensor that gives an excellent sense of wearing and can stably detect weak biological signals, and a method of manufacturing the biological sensor.

Hereinafter, a biological sensor and a method of manufacturing the biological sensor according to an embodiment of the present disclosure will be described. It is to be noted that various elements in the drawings are schematically illustrated for the purpose of understanding the present disclosure and the dimension ratio and the external appearance may be different from the actual ones.

A biological sensor according to an aspect of the present disclosure includes: a sheet having flexibility and/or stretchability; multiple projections that are provided on one of the surfaces of the sheet, and are configured to be brought into contact with a living body to obtain information on the living body. The surfaces of the multiple projections each have conductivity, and the multiple projections are each an elastic structure.

This allows the biological sensor to conform to the shape of the surface of the living body and to deformation of the living body surface due to body motion. Thus, the adhesiveness between the living body surface and the multiple projections can be ensured. Therefore, the biological sensor can stably detect weak biological signals. In addition, since the multiple projections are each an elastic structure, an external force received by the biological sensor can be dispersed. Consequently, uncomfortable sense such as a sense of tightening is not given to a user, and an excellent sense of wearing is provided.

For instance, in the biological sensor, the surfaces of top portions of the multiple projections may include respective conductive patterns, and the conductive patterns may be connected via a conductive pattern on the lateral surfaces of the projections and on the one surface of the sheet.

Thus, the biological sensor can be regarded as a single electrode including the conductive patterns (hereinafter simply referred to as a “detection electrode”) provided in the surfaces of top portions of the multiple projections.

For instance, in the biological sensor, the sheet may include a first sheet and a second sheet provided on the first sheet, and the multiple projections may be projecting portions provided in the second sheet.

In this case, the biological sensor has a structure in which two sheets are stacked, which makes it easy to form spaces enclosed by the two sheets.

For instance, in the biological sensor, a surface of the second sheet opposite to a surface including the multiple projections may be bonded to the first sheet.

Consequently, even when the biological sensor receives an external force, two sheets are unlikely to be displaced.

For instance, the biological sensor may have enclosed spaces between the inner-side surfaces of the multiple projections and the first sheet. In this case, the biological sensor according to the aspect of the present disclosure may have a fluid or an elastomer having flexibility higher than the flexibility of the second sheet, within each of the enclosed spaces. The enclosed spaces may be airtight spaces.

In this manner, the biological sensor has an increased elasticity of the multiple projections by sealing an elastic material in each of the enclosed spaces, and can be flexibly deformed in response to stress from the outside. Thus, the biological sensor can ensure the adhesiveness to the living body surface, and uncomfortable sense such as sense of restraint is unlikely to be given to a user.

For instance, in the biological sensor, each of the multiple projections may have a meandering shape in plan view of the sheet.

In this biological sensor, each of the enclosed spaces also has a meandering shape. Thus, even when a strong pressure is applied to part of one of the projections in the meandering shape, the pressure can be distributed and equalized within the corresponding enclosed space. Therefore, a natural sense of wearing without a local sense of tightening can be obtained.

A method of manufacturing a biological sensor according to an aspect of the present disclosure includes: a conductive pattern formation step for forming a conductive pattern in one of the surfaces of a sheet having flexibility and/or stretchability; and a projection formation step for forming, on the surface on which the conductive pattern is formed, multiple projections that are configured to be brought into contact with a living body to obtain information on the living body. The multiple projections are each an elastic structure.

Thus, it is possible to obtain a biological sensor that gives an excellent sense of wearing and can stably detect weak biological signals.

For instance, in the method of manufacturing the biological sensor, the sheet includes a first sheet and a second sheet provided on the first sheet, in the conductive pattern formation step, the conductive pattern is formed on one of the surfaces of the second sheet, and the projection formation step may include a processing step for forming multiple projections on the one surface of the second sheet, on which the conductive pattern is formed, and a bonding step for bonding the other surface of the second sheet to the first sheet, the other surface being on the opposite to the one surface on which the multiple projections are formed.

In this case, it is easy to form spaces enclosed by two sheets by adopting a structure in which the two sheets are stacked.

For instance, in the method of manufacturing the biological sensor, in the processing step, a recessed portion may be formed in an inner-side surface of each of the multiple projections, and in the bonding step, the second sheet and the first sheet may be bonded, and enclosed spaces may be formed between the inner-side surfaces of the multiple projections and the first sheet.

In this case, the projection formation step may include filling step between the processing step and the bonding step. The filling step is a step for filling a fluid or an elastomer having a flexibility higher than the flexibility of the second sheet, in the inner-side of each of the multiple projections.

In this manner, the biological sensor has an increased elasticity of the multiple projections by sealing an elastic material in the enclosed space, and can be flexibly deformed in response to stress from the outside. Thus, it is possible to obtain a biological sensor that can ensure the adhesiveness to the living body surface, and uncomfortable sense such as sense of restraint is unlikely to be given to a user.

Hereinafter, an embodiment of the present disclosure will be specifically described with reference to the drawings. The biological sensor according to the embodiment of the present disclosure measures a signal on a living body (that is, a biological signal), such as a bioelectric potential, and obtains information on the living body (for instance, one or multiple pieces of vital signs information, such as electrocardiogram, brain waves, and electromyogram).

It is to be noted that each of the embodiments described below illustrates a comprehensive or specific example. The numerical values, shapes, materials, structural components, the arrangement and connection of the structural components, steps, the sequence of the steps illustrated in the following embodiments are mere examples, and are not intended to limit the scope of the present disclosure. Therefore, among the structural components in the subsequent embodiments, components not recited in any one of the independent claims which indicate the most generic concept are described as arbitrary structural components. It is to be noted that the respective figures are schematic diagrams and are not necessarily precise illustrations. In the respective figures, the same reference sign is given to substantially identical components, and redundant description is omitted or simplified.

### First Embodiment

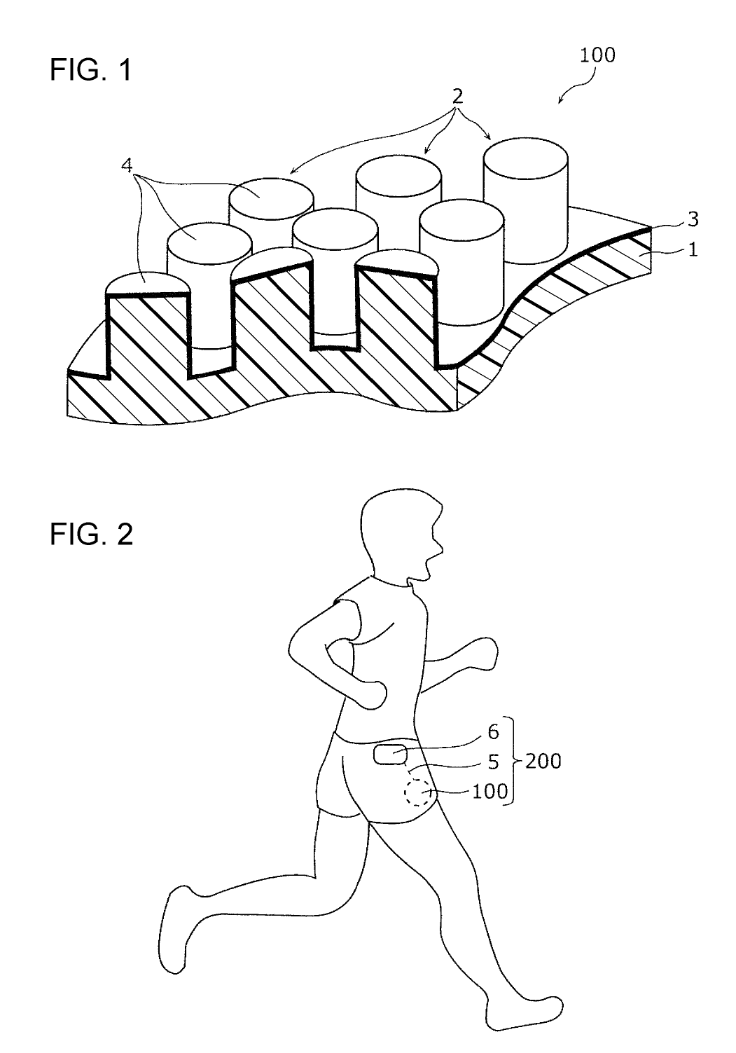

FIG. 1 is a sectional perspective view schematically illustrating a biological sensor 100 according to a first embodiment. As illustrated in FIG. 1, the biological sensor 100 according to the first embodiment includes multiple projections 2 disposed with predetermined intervals on one of the surfaces of a sheet 1 having flexibility and/or stretchability. The surfaces of top portions of the multiple projections 2 include respective conductive patterns 3, and the respective multiple conductive pattern 3 are connected via a conductive pattern 3 on the lateral surfaces of the projections 2 and on the one surface of the sheet 1. In this manner, the lateral surfaces of the projections 2 and the one surface of the sheet 1 including the projections 2 are each provided with the conductive pattern 3. Thus, biological signals obtained from the top portions of the multiple projections 2 can be electrically drawn out to the outside. Also, the multiple projections 2 are each an elastic structure that can be flexibly deformed in response to an external force applied to the multiple projections 2. In this manner, the multiple projections 2 are each an elastic structure which allows the multiple projections 2 to conforms to the shape of the living body surface and to deformation of the living body surface due to body motion. Thus, it is possible to ensure the adhesiveness between the living body surface and the conductive pattern 3 (hereinafter referred to as a “detection electrode 4”) included in each top portion of the multiple projections 2. Thus, the biological sensor 100 can stably ensure the electrical connection between the living body surface and the detection electrode 4.

FIG. 2 is a diagram illustrating an example of use of the biological sensor 100 according to the first embodiment. FIG. 2 illustrates an example of measuring biological signals when a user wears a wearable biological signal measurement device 200 configured using the biological sensor 100 according to the first embodiment. In this example, the biological sensor 100 measures muscle potentials of a thigh.

The wearable biological signal measurement device 200 includes the biological sensor 100 that detects a biological signal, a signal processing circuit unit 6 that converts the obtained biological signal to a digital signal and wirelessly transmits the digital signal to an external terminal, and a wire 5 and a connector that electrically connect the biological sensor 100 and the signal processing circuit unit 6. In the example of FIG. 2, the biological sensor 100 is disposed inside sport trousers, and the biological sensor 100 is slightly pressed against the thigh due to the stretchability of the sport trousers. Thus, the biological sensor 100 and the living body surface are brought into intimate contact with each other. The signal processing circuit unit 6 is fixed to a hip by a belt or the like, and the biological sensor 100 and the signal processing circuit unit 6 are connected by the wire 5.

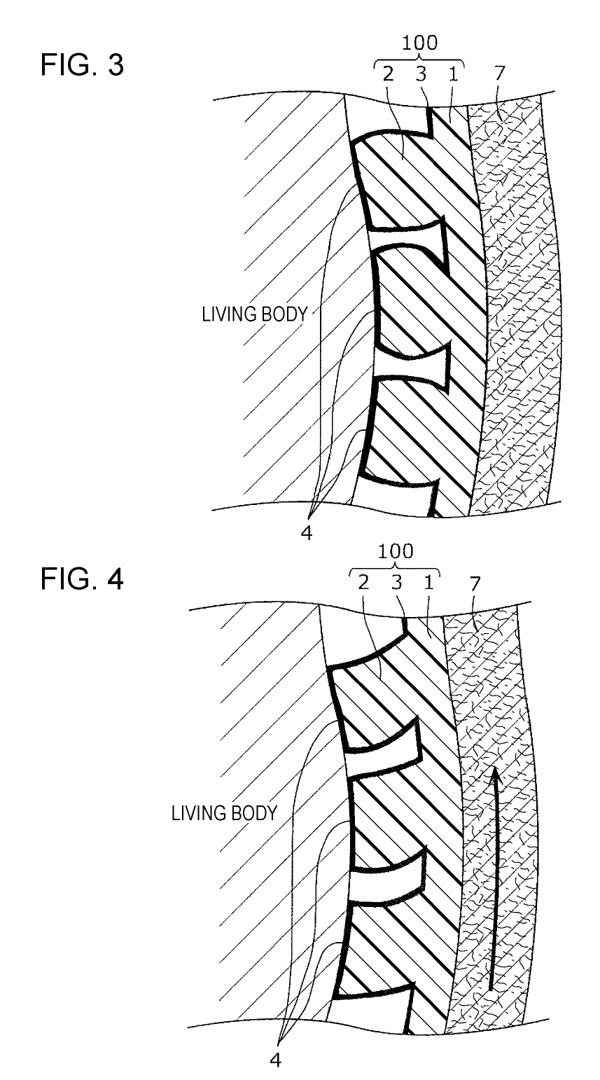

FIG. 3 is a sectional view schematically illustrating the biological sensor 100 when a user wears clothes equipped with the biological sensor 100 in the example of use of FIG. 2.

As illustrated in FIG. 3, cloth 7 of the sport trousers and the sheet 1 of the biological sensor 100 are bonded and fixed. As described above, the biological sensor 100 includes multiple projections 2 on one of the surfaces of the sheet 1 having flexibility and/or stretchability. The multiple projections 2 are each an elastic structure. In the biological sensor according to the present disclosure, the sheet 1 has flexibility and/or stretchability. The sheet 1 may have both flexibility and stretchability. In this embodiment, the sheet 1 has flexibility and stretchability.

The material of the sheet 1 is not particularly restricted as long as the material provides flexibility and/or stretchability. For instance, the material may be a resin material. Thus, the biological sensor 100 can conform to the complicated shape of the living body surface and to deformation of the living body surface due to body motion.

The resin material includes an elastomeric material, and a rubber material. These resin materials may be used singly or may be used in a combination or two or more types.

As described above, in the biological sensor 100 according to this embodiment, the sheet 1 has flexibility. Since the sheet 1 has flexibility, the biological sensor 100 also has flexibility. In addition, since the sheet 1 has stretchability, the biological sensor 100 easily fits to the shape of the living body surface, and easily follows the motion of the living body. Therefore, connection reliability between the living body surface and the detection electrode 4 included in each top portion of the multiple projections 2 can be improved.

It is to be noted that the stretching direction of the sheet 1 may be a two-dimensional direction in the plane of the sheet 1, or a three-dimensional direction further including a perpendicular direction to the sheet 1. Thus, even when a predetermined area of the living body has a three-dimensionally complicated shape, the biological sensor 100 can be fitted and brought into intimate contact with the shape of the living body surface. In addition, the biological sensor 100 can be stretched or contracted by conforming to a specific portion of the living body surface, which is stretched or contracted due to a motion of the living body.

Also, since the sheet 1 has flexibility, the biological sensor 100 can conform to deformation of sport trousers. A material having excellent stretchability, such as a knitting material is used as the cloth 7 of the sport trousers. Since the sheet 1 has flexibility and stretchability, the biological sensor 100 has better conforming performance to the cloth 7 of the sport trousers, as compared with the case where the sheet 1 has flexibility or stretchability. Since the cloth 7 of the sport trousers conforms to the shape of the living body surface and to deformation of the living body surface due to body motion, the sheet 1 has better conforming performance to the cloth 7 of the sport trousers. Thus, the biological sensor 100 also has better conforming performance to the living body surface.

The multiple projections 2 provided on the main surface of the sheet 1 are pressed against the surface of skin due to the stretchability of the sport trousers, with the conductive pattern 3 in contact with the surface of skin. Thus, the conductive pattern 3 is electrically connected to the living body surface.

Here, the biological sensor 100 is compared with a dry electrode using conductive fiber cloth in related art. The dry electrode in related art is a cloth-like electrode formed by knitting fiber of a twisted thread obtained by twisting a fine conductive thread. For this reason, when the dry electrode is brought into contact with the living body surface, each piece of fine conductive thread forming the cloth is electrically connected to the living body surface. Thus, even when the entire cloth surface is in contact with the living body surface, the contact is made by a set of electrical point contact between each conductive thread included in the cloth and the living body surface. Thus, a contact resistance is high and signals from the biological sensor are unlikely to be stabilized. In order to reduce the contact resistance and to stabilize the signals from the biological sensor, it is necessary to increase the restraining pressure of clothes which restrains the living body surface to press the entire surface of the cloth inside the clothes against the living body surface. In other words, for clothes using a dry electrode in related art, the dry electrode needs to be deformed by a high restraining force so that more conductive threads forming the cloth of the clothes are brought into direct contact with the living body surface. Since intimate contact between the dry electrode and the living body surface is attempted to be made by a high restraining force for clothes using a dry electrode in related art, sense of wearing when the clothes are worn, such as sense of tightening is worsened.

In the biological sensor 100 according to this embodiment, the surfaces of top portions of the multiple projections 2 include respective conductive patterns 3 (i.e., detection electrodes 4). Thus, in contrast to the point contact of the dry electrode in related art, the conductive patterns 3 each come into surface contact with the living body surface. Therefore, in contrast to the biological sensor using a dry electrode in related art, the contact resistance between the living body surface and the detection electrodes 4 is reduced. In order to obtain a stable low contact resistance, it is necessary to ensure a state that allows the entire surfaces of the detection electrodes 4 to come into contact with the living body surface. In the biological sensor 100 according to this embodiment, the multiple projections 2 are each an elastic structure. Thus, contact state between the living body surface and the detection electrodes 4 can be ensured even with a low restraining force.

The elastic structure is a structure having such property that when the structure receives an external force from an object, the structure is easily deformed, conforming to the shape of the object, and when the external force is removed, the structure attempts to restore at least the original shape. Therefore, not only a mass of a simple elastic material such as rubber or resin, but also an object that structurally exhibits the above-described property, such as a bag in which gas or fluid is sealed, for instance, a balloon is also included in the elastic structure.

As illustrated in FIG. 3, the multiple projections 2 are each an elastic structure. Thus, when only slightly pressed, each structure is deformed in a barrel shape, due to the stretchability of clothes. In this situation, the surface of each top portion of the projection 2 is pressed against the living body surface, and the entire surface is brought into intimate contact with the living body surface. Therefore, the conductive pattern 3 included in each top portion of the multiple projections 2, that is, the detection electrode 4 can ensure the adhesiveness with the living body surface. Also, since the multiple projections 2 are each an elastic structure, a pressing pressure due to the stretchability of clothes can be dispersed, and uncomfortable sense such as a sense of tightening is unlikely to be felt by a user.

Also, for the dry electrode in related art, an extremely strong pressing force is necessary to increase the contact area between the living body surface and each piece of conductive thread included in the dry electrode. In contrast, in the biological sensor 100 according to this embodiment, the multiple projections 2 are each an elastic structure, and there is space between adjacent projections 2 among the multiple projections 2. Thus, an extremely strong fastening force as in the case of the dry electrode in related art is unnecessary. When the biological sensor 100 according to this embodiment receives an external force, the multiple projections 2 disperse the external force into a force which presses against the living body surface and a force which escapes in a direction of the lateral surface of each projection 2, that is, a direction of space between projections 2. Therefore, even when receiving a low pressing pressure, the multiple projections 2 can disperse the pressure into a force which presses against a surface in contact with the living body surface, and a force which escapes in a direction of the lateral surface of each projection 2. The multiple projections 2 disperse the external force received by the biological sensor 100, which can ensure stable contact with the living body surface by an appropriate fastening force without giving uncomfortable sense such as a sense of tightening to a user.

Also, since the multiple projections 2 are provided, corresponding projections 2 can conform to and maintain contact with local depressions and projections or slope on the living body surface. Consequently, each projection 2 can be deformed while following the motion of the living body surface. Thus, the conductive pattern 3 included in the surface of each top portion of the projections 2, that is, the detection electrode 4 can conform to the changing shape of the living body surface to come into surface contact with the living body surface.

FIG. 4 is a sectional view schematically illustrating the biological sensor 100 when a user wears clothes equipped with the biological sensor 100 and performs an activity in the example of use of FIG. 2. The biological sensor 100 according to this embodiment can achieve stable biological sensing by reducing displacement of the detection electrode 4 from the living body surface. As illustrated in FIG. 4, when a user wears a sportswear clothes equipped with the biological sensor 100 and performs an activity, the cloth of the sportswear is pulled according to a body motion, and accordingly, the biological sensor 100 is also pulled and receives an external force in a direction in which the electrode is displaced. In this situation, when the clothes have an extremely strong fastening force, the clothes are unlikely to be pulled according to a body motion, and displacement of the electrode from the living body surface is unlikely to occur. However, uncomfortable sense such as a sense of tightening is given to a user. In contrast, when a dry electrode in related art is used for clothes having a low fastening force not giving uncomfortable sense to a user, the position of the electrode is displaced from the living body surface, and the voltage level of the biological sensor causes pulsed noise, resulting in a situation in which normal biological sensing cannot be achieved. In particular, in the case of an activity with a vigorous body motion, such as sports, the electrode is likely to be displaced, and measurement of a biological signal may be difficult. However, in the biological sensor 100 according this embodiment, the multiple projections 2 are each an elastic structure which can be flexibly deformed in response to an applied stress. Thus, even for a stress which causes a positional displacement of the electrode, each projection 2 can be deformed in a shear distortion direction which is diagonally inclined, and can serve to resist against displacement of the electrode from the living body surface.

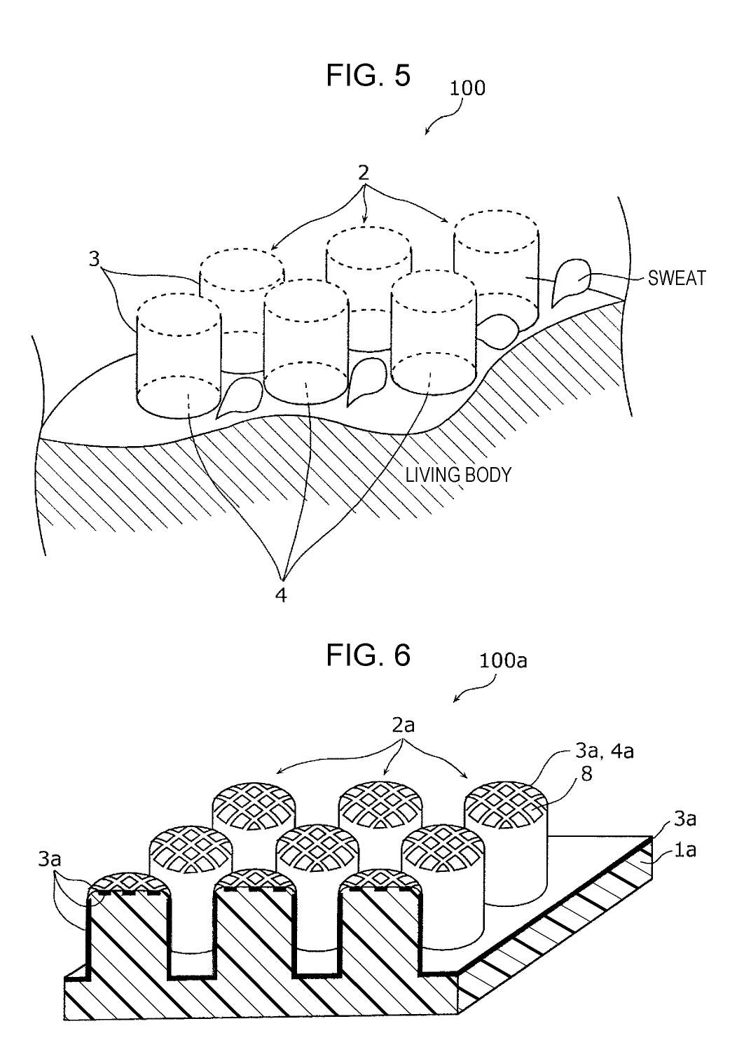

FIG. 5 is a diagram explaining the biological sensor 100 and the state of the surface of a user's body while the user is performing an activity. Since the biological sensor 100 has space between any adjacent projections 2 among the multiple projections 2, even when a user sweats in an intense activity such as sports, drops of sweat can be discharged. In a gel electrode in related art, the entire electrode surface comes into intimate contact with the living body surface, and drops of sweat are collected between the electrode and the living body surface. Thus, the effect on the skin needs to be considered. In contrast, in the biological sensor 100 according to this embodiment, although the conductive pattern 3 in the surface of each of the projections 2, that is, the detection electrode 4 comes into intimate contact with the living body surface on the entire surface, space between any adjacent projections 2 is provided. Thus, a flow path for flowing out drops of sweat can be ensured. Therefore, the biological sensor 100 can reduced the effect of sweat on the skin, and can maintain comfortable sense of wearing.

Hereinafter, a method of manufacturing the biological sensor 100 according to the first embodiment will be described.

First, in the projection formation step, multiple projections 2 are formed by bonding multiple members on one of the surfaces of a sheet 1. Thus, a structure including the sheet 1 and the multiple projections 2 is formed. The multiple members are molded in projecting shapes from a resin material, such as an elastomer. It is to be noted that a resin material, such as an elastomer may be cast in a mold having a shape integrating the sheet 1 and the multiple projections 2 so that the sheet 1 and the multiple projections 2 are integrally formed.

Subsequently, in the conductive pattern formation step, a conductive pattern 3 is formed so as to completely cover the entire surface, where the multiple projections 2 are formed, of the structure including the sheet 1 and the multiple projections 2. In other words, the conductive pattern 3 is formed by printing on the surfaces of the multiple projections 2, and a portion in which the multiple projections 2 are not formed, the portion being of the one surface of the sheet 1.

It is to be noted that instead of performing the above-described conductive pattern formation step, in the projection formation step, the biological sensor 100 may be manufactured by using a resin material having conductivity, such as an elastomer, as the material of the sheet 1 and the multiple projections 2. In this case, the sheet 1 has an insulating sheet or an insulating film on a surface opposite to the surface on which the multiple projections 2 are formed.

### Modification of First Embodiment

FIG. 6 is a sectional perspective view schematically illustrating a biological sensor 100a according to a modification of the first embodiment. The biological sensor 100a is different from the biological sensor 100 of the first embodiment in the following points, and is the same as the biological sensor 100 in the other points. The biological sensor 100a includes a conductive pattern 3a in a shape having one or multiple openings 8 on the surface of each top portion of the multiple projections 2a. The shape of the conductive pattern 3a is a lattice shape, for instance. The conductive pattern 3a of each top portion of the multiple projections 2a is a detection electrode 4a of the biological sensor 100a. Respective multiple conductive patterns 3a of the top portions of the multiple projections 2a are electrically connected to each other via the surfaces of the lateral sides of the multiple projections 2 and the conductive patterns 3a on one surface of the sheet.

In the biological sensor 100a according to this modification, the conductive pattern 3a in a shape having multiple openings 8 is provided on the surface of each top portion of multiple projections 2a. Thus, the surface of each projection 2a is exposed from the openings 8. Since each projection 2a is composed of an elastic material, part of the projection 2a comes into direct contact with the living body surface through the openings 8. Thus, part of the projection 2a exposed through the openings 8 serves as slip resistance. Here, an elastic material is an elastomer material such as a silicone resin or an urethane resin, and is so-called a rubber-like material. Therefore, the projection 2a composed of an elastic material has an extremely higher frictional force against the living body surface than the surface of the conductive pattern 3a, and part of the projection 2a exposed through the openings 8 serves as slip resistance.

The conductive pattern 3a is formed by coating and curing a conductive paste on the surface of the projection 2a and a surface of the sheet 1a, the surface provided with the projection 2a, the conductive paste being obtained by kneading, for instance, an elastomer material such as silicone or urethane, and a conductive filler such as silver powder. Thus, flexibility or stretchability and conductivity of the conductive pattern 3a both can be achieved. The conductive pattern 3a contains an elastomer material, and contains the conductive filler with a high volume ratio to ensure conductivity. For this reason, the frictional force on the surface, in contact with a living body surface, of the conductive pattern 3a is less than the frictional force of the elastomer material. In this case, depending on the fastening force of clothes, when a force is applied to the biological sensor 100a in a direction in which the electrode is displaced from the living body surface, the conductive pattern 3a included in the surface of the top portion of the projection 2a may slide and the position of the electrode may be displaced. Thus, in the biological sensor 100a according to this modification, in order to reduce the positional displacement of the electrode, openings 8 are provided in the conductive pattern 3a included in the surface of the top portion of the projection 2a. Consequently, as described above, it is possible to achieve the detection electrode 4a of the biological sensor 100a that is unlikely to be displaced from the living body surface.

The biological sensor 100a according to this modification differs from the biological sensor 100 in that the conductive pattern 3a having the openings 8 is included in the surface of each top portion of the multiple projections 2a. Thus, in a method of manufacturing the biological sensor 100a according to this modification, in the conductive pattern formation step, the conductive pattern 3a is formed so that the openings 8 are formed in the surface of each top portion of the multiple projections 2a. A resin material such as an elastomer material having an insulating property rather than a conductive material is used as the material of the sheet 1a and the multiple projections 2a. Except for these points, the method of manufacturing the biological sensor 100a is the same as the method of manufacturing the biological sensor 100 according to the first embodiment.

### Second Embodiment

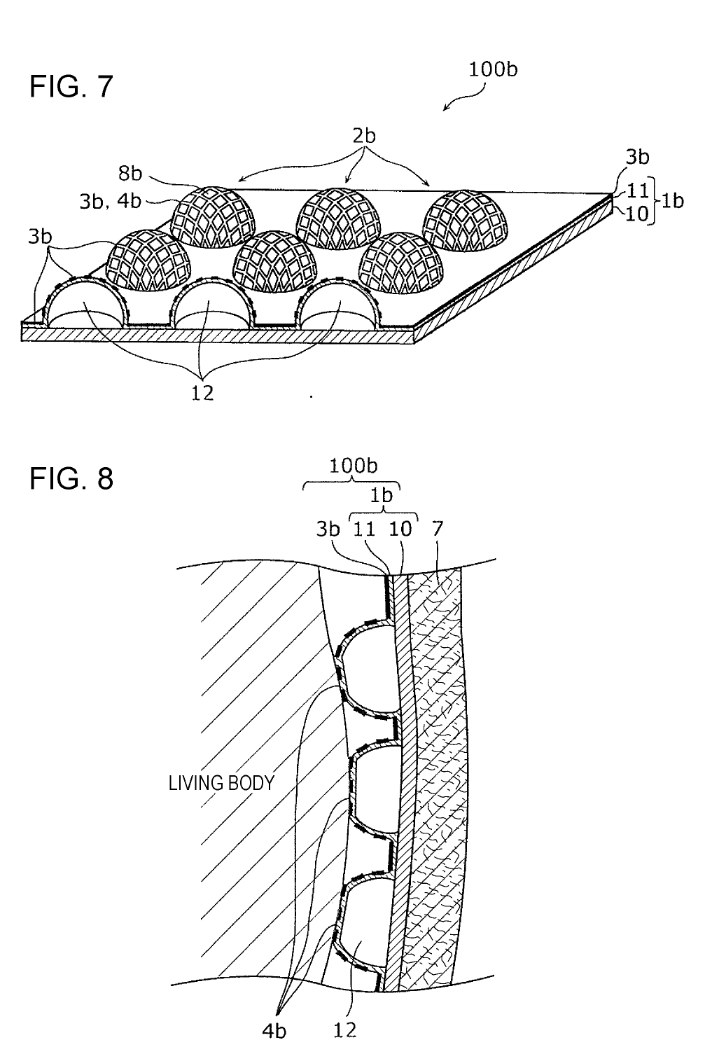

FIG. 7 is a sectional perspective view schematically illustrating a biological sensor 100b according to a second embodiment. Unlike the above-described biological sensors 100 and 100a, in the biological sensor 100b according to the second embodiment, a sheet 1b having flexibility and stretchability includes a first sheet 10, and a second sheet 11 provided on the first sheet 10. The second sheet 11 includes multiple projections 2b.

As illustrated in FIG. 7, similarly to the biological sensors 100 and 100a described above, in the biological sensor 100b, multiple projections 2b are provided with predetermined intervals on one surface of the sheet 1b having flexibility and stretchability. Specifically, the second sheet 11 has a first surface facing the first sheet 10, and a second surface opposite to the first surface, and the second surface has multiple projecting shapes. The biological sensor 100b includes a conductive pattern 3b in a shape having openings 8b on the surface of each of the multiple projections 2b. The conductive pattern 3b of each surface of the multiple projections 2b is a detection electrode 4b. Multiple detection electrodes 4b are electrically connected to each other via the conductive patterns 3b on the second surface of the second sheet 11. The second sheet 11 and a member including the conductive patterns 3b are each an example of the second sheet of the present disclosure.

The first surface of the second sheet 11 may be bonded to the first sheet 10. The material of the first sheet 10 is the same as the material of the sheet 1 in the first embodiment described above. Similarly to the first sheet 10, the material of the second sheet 11 is not particularly restricted as long as the material has flexibility and/or stretchability. The material of the second sheet 11 may be, for instance, an elastomer material such as urethane and silicone, or a synthetic rubber material.

The multiple projections 2b are produced as follows. First, a single sheet, which is to be the second sheet 11, is set in a mold, and respective portions corresponding to the multiple projections 2b are molded in projecting shapes. Subsequently, in the sheet in which the projecting shapes are formed, a portion other than the projecting shapes, is bonded to the first sheet 10. Specifically, the portion surrounding the multiple projecting shapes in the first surface of the second sheet 11 is bonded to the first sheet. In this embodiment, enclosed spaces 12 are formed between the inner-side surfaces of the multiple projections 2b and the first sheet. It is to be noted that the biological sensor 100b may have a fluid or an elastomer having flexibility higher than the flexibility of the second sheet, in each of the enclosed spaces 12. In this embodiment, air is sealed in each of the enclosed spaces 12 and the multiple projections 2b can be flexibly deformed in response to an external stress just like a balloon. Thus, in the biological sensor 100b according to this embodiment, each of the multiple projections 2b are not only elastic as the material, but also have structurally elastic characteristic.

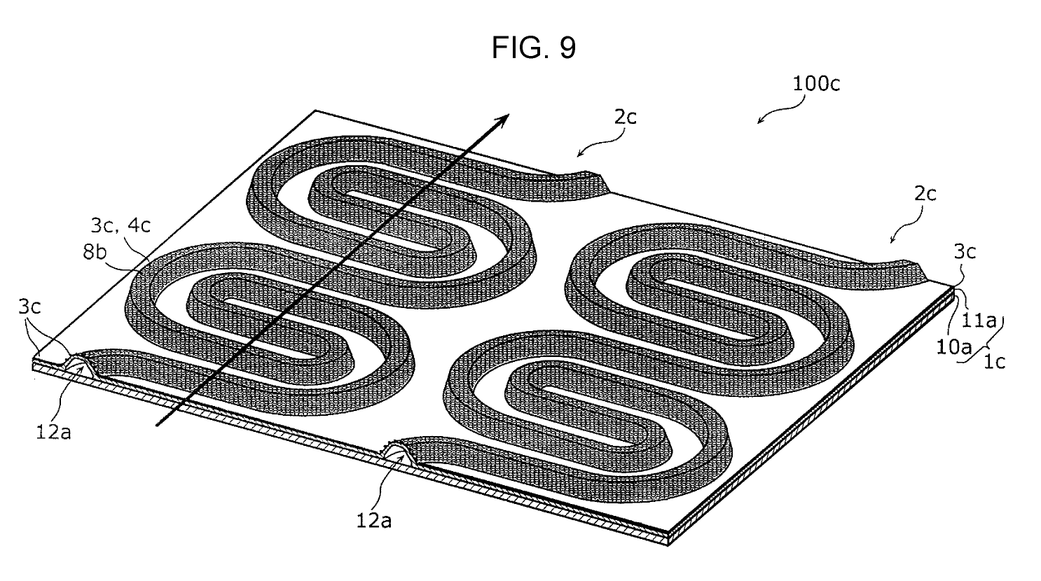

FIG. 8 is a sectional view schematically illustrating the manner in which the biological sensor 100b according to the second embodiment is in intimate contact with the living body surface. In a state where a stress is not applied to the biological sensor 100b, as illustrated in FIG. 7, the multiple projections 2b each have a hemispherical convex shape, and the conductive pattern 3b included in the surface of each projection 2b conforms to the surface shape of the projection 2b and also has a convex shape. When a stress is applied to the biological sensor 100b, as illustrated in FIG. 8, only a slight press of the multiple projections 2b against a living body surface causes the top portion of each projection 2b to deform like a balloon to conform to the shape of the living body surface. Thereby, the conductive pattern 3b is brought into contact with the shape of the living body surface. In contrast, in a dry electrode in related art, as described above, the contact area with the living body surface is small, and the contact resistance is high. However, in the biological sensor 100b according to this embodiment, the multiple projections 2b conform to and come into surface contact with the living body surface. Thus, the contact area with the living body surface can be increased, and the contact resistance can be lowered. Therefore, even when the shape of the living body surface is deformed due to a body motion, the biological sensor 100b can perform stable biological sensing by conforming to the living body surface all the time.

This embodiment gives an example in which the multiple projections 2b each have a hemispherical convex shape, for instance. Since the multiple projections 2b each have a convex shape, the contact with the living body surface conforming to depressions and projections of the living body surface is ensured, and portion which is in relatively tight contact with the living body surface and a portion which is in relatively loose contact with the living body surface are exist. Consequently, the biological sensor 100b reduces the difference between pressures applied to the living body surface, as compared with the biological sensors 100 and 100a described above, and can reduce uncomfortable sense when the clothes are worn. In this embodiment, when the projections 2b in convex shapes come into contact with the living body surface, only the top portions in convex shapes are first deformed, and the multiple projections 2b start to conform to the shape on the living body surface. Subsequently, as a stress pressing the biological sensor 100b against the living body surface is increased, the portion surrounding the top portions in convex shapes is also deformed, and the multiple projections 2b have a larger area which conforms to the living body surface. In this manner, in the biological sensor 100b, the multiple projections 2b can have a larger contact area with the living body surface, as compared with the biological sensors 100 and 100a described above. Here, the pressure actually applied to the living body surface is a value obtained by dividing a stress which presses the biological sensor against the living body surface by the contact area between the biological sensor and the living body surface. Thus, even when the biological sensor 110b is pressed against the living body surface by a locally strong stress at a portion, the area that conforms to the living body surface is increased to reduce the sensitivity of a user for a pressure difference so that the locally strong stress is not likely to be felt by a user.

Although this embodiment illustrates an example in which air is sealed in the enclosed space 12, liquid such as silicone oil may be sealed in the enclosed space 12, for instance, and an elastomer material having flexibility higher than the flexibility of the second sheet 11 may be sealed in the enclosed space 12. Thus, the same effect as in this embodiment is obtained.

A method of manufacturing the biological sensor 100b according to the second embodiment is the same as the method of manufacturing the biological sensor 100d according to the later-described second modification except that the print pattern of the conductive pattern 3b is different. Thus, a description of the method of manufacturing the biological sensor 100b according to the second embodiment is omitted here.

### First Modification of Second Embodiment

FIG. 9 is a sectional perspective view schematically illustrating a biological sensor 100c according to a first modification of the second embodiment. In the biological sensor 100c according to this modification, each of multiple projections 2c has a meandering shape in a plan view of the sheet 1c.

As illustrated in FIG. 9, similarly to the above-described biological sensors 100 to 100b, in the biological sensor 100c, multiple projections 2c are provided with predetermined intervals on one surface of the sheet 1c having flexibility and stretchability. Specifically, a second sheet 11a has a first surface facing a first sheet 10a, and a second surface opposite to the first surface, and the second surface has multiple projecting shapes. The biological sensor 100c includes a conductive pattern 3c in a shape having openings 8b on the surfaces of multiple projections 2c. The conductive pattern 3b of each surface of the multiple projections 2c is a detection electrode 4c. Multiple detection electrodes 4c are electrically connected to each other via the conductive patterns 3c on the second surface of the second sheet 11a.

In the biological sensor 100b according to the second embodiment, as illustrated in FIG. 7, independent convex-shaped multiple projections 2b are arranged with predetermined intervals two-dimensionally in a long length direction and a short length direction of the sheet 1b. In contrast, in the biological sensor 100c according to this modification, as illustrated in FIG. 9, each projection 2c has a meandering continuous shape extending from one end of the sheet 1c in a long length direction or a short length direction of the sheet 1c.

As explained above, the projection 2c has a continuously extending shape. However, when depressions and projections of the projection 2c in the meandering shape in the direction of the arrow of FIG. 9 are viewed, the depressions and projections are arranged with predetermined intervals. Thus, similarly to the multiple projections 2 to 2b of the above-described biological sensors 100 to 100b, the projection 2c in a meandering shape can be flexibly deformed to maintain contact with the depressions and projections of the living body surface, and can conform to the living body surface deformable due to a body motion.

Also, the multiple projections 2c in meandering shapes may have enclosed spaces 12a between the inner-side surfaces of the multiple projections 2c and the first sheet 10a. Specifically, the portion surrounding the multiple projecting shapes in the first surface of the second sheet 11 a is bonded to the first sheet. Thus, the enclosed space 12a defined by the first surface and the first sheet 10a is present within each of the multiple projections. Although this modification illustrates an example in which air is sealed in the enclosed space 12a, a gas such as an inactive gas, or a fluid such as liquid or gel may be sealed in the enclosed space 12a, and the biological sensor 100c may have an elastomer having flexibility higher than the flexibility of the second sheet 11a. Thus, even when a high pressure is applied to part of the projection 2c in a meandering shape, a fluid moves in the enclosed space 12a of the projection 2c in a meandering shape. Thus, the pressure applied to the living body surface can be equalized within the projection 2c in a meandering shape. Therefore, a more natural sense of wearing without a sense of locally tightening can be obtained, as compared with the biological sensor 100b according to the second embodiment. The second sheet 11a and a member including the conductive patterns 3c are an example of the second sheet of the present disclosure.

A method of manufacturing the biological sensor 100c according to the first modification of the second embodiment is the same as the method of manufacturing the biological sensor 100d according to the later-described second modification except that the print pattern of the conductive pattern 3c is different. Thus, a description of the method of manufacturing the biological sensor 100c according to the first modification is omitted here.

### Second Modification of Second Embodiment

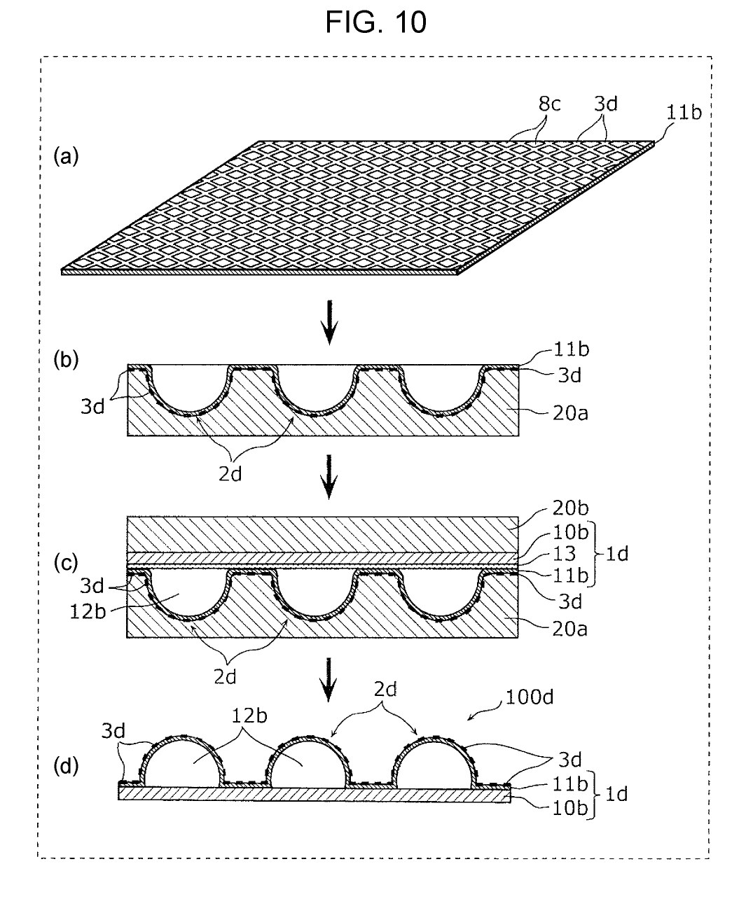

FIG. 10 is a diagram explaining a method of manufacturing a biological sensor 100d according to a second modification of the second embodiment.

Unlike the above-described biological sensors 100b and 100c, in the biological sensor 100d according to this modification, a conductive pattern 3d having openings 8c is included in the entire surface, in which projections 2d are provided, of the second sheet 11b. Other portions of the biological sensor 100d according to the second modification are the same as those of the biological sensor 100b according to the second embodiment. It is to be noted that in the second modification, a sheet 1d includes a first sheet 10b, a bonding layer 13, and the second sheet 11b.

Hereinafter, a method of manufacturing the biological sensor 100d according to the second modification of the second embodiment will be described.

The biological sensor 100d according to the second modification is manufactured by using the first sheet 10b and the second sheet 11b. In the processing step illustrated in FIG. 10(b), multiple projections 2d are provided in the surface that includes the conductive pattern 3d of the second sheet 11b. In this modification, a description is given by way of an example in which the shapes of multiple projections 2d are molded in the second sheet 11b using a mold.

### (Conductive Pattern Formation Step)

First, as illustrated in FIG. 10(a), conductive paste is printed, for instance, in a lattice pattern on one surface of a single sheet which is to be the second sheet 11b, and the conductive pattern 3d having the openings 8c is formed. Here, a polyurethane sheet having flexibility and stretchability is used as a sheet to be the second sheet 11b. A paste obtained by kneading an urethane resin having stretchability and silver powder is used as the conductive paste. In this manner, both a sheet to be the second sheet 11b and the conductive pattern 3d have stretchability.

### Processing Step

Subsequently, as illustrated in FIG. 10(b), a structure including a sheet to be the second sheet 11b and the conductive pattern 3d is inverted and disposed so that the conductive pattern side faces a mold 20a. Subsequently, the structure is deformed and fixed to conform to the shape of the mold 20a, and multiple projections 2d are molded. Thus, the second sheet 11b having projecting shapes is formed. The second sheet 11b and a member including the conductive pattern 3d are an example of the second sheet of the present disclosure. In the processing step, the multiple projections 2d may be molded by pressing the structure, which includes a sheet to be the second sheet 11b and the conductive pattern 3d, from above against recessed portions of the mold 20a with another mold for pressing. Alternatively, the structure may be pressed from above against the recessed portions by air pressure or fluid pressure to mold the multiple projections 2d. Alternatively, a path for vacuum suction may be provided in the recessed-portions of the mold 20a, and the multiple projections 2d may be molded by performing vacuum suction so that the structure conforms to the shape of the mold 20a. In this case, since a material having excellent stretchability is used for both a sheet to be the second sheet 11b and the conductive pattern 3d, the multiple projections 2d, which conform to the shape of the mold 20a, can be easily molded.

### Bonding Step

Subsequently, as illustrated in FIG. 10(c), the first sheet 10b is stacked and pressed by a mold 20b on a surface opposite to the surface of the second sheet 11b, on which the multiple projections 2d are molded, and the second sheet 11b is bonded to the first sheet 10b. More specifically, in the second sheet 11b, the area surrounding the portions where respective projections 2d are molded is bonded to the first sheet 10b via the bonding layer 13. Thus, the enclosed spaces 12b are formed inside the multiple projections 2d.

In the bonding step, a thermoplastic urethane sheet serving as the bonding layer 13 is interposed between the second sheet 11b and the first sheet 10b, and is heated while being pressurized by the mold 20a and the mold 20b. Thus, the bonding layer 13 is softened then cooled, and thereby the second sheet 11b and first sheet 10b can be bonded. Alternatively, the second sheet 11b and first sheet 10b may be heat-sealed without using the bonding layer.

Consequently, as illustrated in FIG. 10(d), the biological sensor 100d having the enclosed spaces 12b can be manufactured. It is to be noted that the bonding layer 13 is not illustrated in FIG. 10(d).

Although in the aforementioned manufacturing method, an example, in which air is sealed in the enclosed spaces 12b formed in the bonding step, has been described, in another aspect, a fluid or an elastomer having flexibility higher than the flexibility of the second sheet 11b may be sealed in the enclosed spaces 12b as described above. In this aspect, after the processing step, filling step is performed for filling liquid or a resin material in recessed portions corresponding to the enclosed spaces 12b of the multiple projections 2d, then the bonding step is performed for stacking the first sheet 10b on the second sheet 11b. In this manner, silicone oil or a flexible urethane resin can be sealed in the enclosed spaces 12b.

The method of manufacturing the biological sensor 100d according to the second modification of the second embodiment has been described above. In the already described biological sensor 100b according to the second embodiment, the conductive pattern 3b having the openings 8a is formed only on the surfaces of multiple projections 2b. Also, in the biological sensor 100c according to the first modification of the second embodiment, the conductive pattern 3c having the openings 8b is formed only on the surfaces of multiple projections 2c. Therefore, the methods of manufacturing the biological sensors 100b, 100c according to the second embodiment and the second modification of the second embodiment differ from each other in that the shapes of the conductive patterns 3b, 3c are only different.

In the second embodiment and the first modification of the second embodiment, the second sheet 11 or 11a and the first sheet 10 or 10a may be bonded by heat-sealing or by using the bonding layer 13. Also when the bonding layer 13 is used, it can be said that the enclosed space within each projection 2b, 2c, 2d is defined by the first surface of the second sheets 11, 11a, 11b and the first sheets 10, 10a, 10b, respectively. Therefore, when the conductive pattern to be formed is changed in the conductive pattern formation step in the manufacturing method in the second modification of the second embodiment, the biological sensors 100b, 100c according to the second embodiment and the first modification of the second embodiment can also be manufactured in the same manner as in this manufacturing method. For this reason, individual description is omitted.

Although the biological sensor and the method of manufacturing the biological sensor according to the present disclosure have been described based on the embodiments above, the present disclosure is not limited to these embodiments. An embodiment to which various alterations which will occur to those skilled in the art are made to the embodiments, and another embodiment constructed by combining part of the components in the embodiments without departing from the spirit of the present disclosure are also included within the scope of the present disclosure.

Also, in the second embodiment or the first modification described above, instead of the conductive patterns 3b, 3c having the openings only on the surfaces of multiple projections, conductive patterns having openings in the entire living body sides of the biological sensors 100b, 100c may be formed. Alternatively, a conductive pattern not having an opening may be formed as in the first embodiment. Alternatively, a conductive pattern having openings only in part of the surface of each projection may be formed as in the modification of the first embodiment. Also, in the second embodiment or the first modification described above, instead of the second sheets 11, 11a, a second sheet having conductivity may be used. Thus, the step of forming the conductive patterns 3b, 3c may be omitted. In this case, the second sheet having conductivity is an example of the second sheet of the present disclosure.

It is to be noted that the conductive pattern having openings may be a stripe shape, a zigzag shape, or a spiral shape.

Also, in the biological sensors according to the present disclosure, the shape of each projection may be a polygonal pillar shape, a pyramid shape, a tapered shape, a dome shape, a hanging bell shape, a substantially spherical shape, or a semi-cylindrical shape.

It is to be noted that all of the shapes of projections may not be the same, and different shapes may be combined. The intervals between projections may not be uniformly spaced.

The biological sensors according to the present disclosure can stably detect weak biological signals with high sensitivity. Thus, the biological sensors may be utilized as sensors used in biological signal measurement devices that measure bioelectric signals, such as muscle potentials, brain waves, and cardiac potentials. Also, the biological sensors according to the present disclosure provides an excellent sense of wearing. Thus, the biological sensors may be utilized as sensors used in wearable biological signal measurement devices used for monitoring biological sensing information in daily life or sports activity, for instance, sensors mounted on a supporter, an underwear, and a sportswear.

What is claimed is:

1. A biological sensor comprising:
a first sheet that has flexibility and/or stretchability; and
a second sheet that has flexibility and/or stretchability, the second sheet having a first surface facing the first sheet, and a second surface opposite to the first surface,
wherein the second surface has a plurality of projections that are configured to be brought into contact with a living body to obtain information on the living body,
in the second surface, at least part of each of the plurality of projections has conductivity,
in the first surface, a portion surrounding each of the plurality of projections is bonded to the first sheet, and
within each of the plurality of projections, an enclosed space defined by the first surface of the second sheet and the first sheet is present.

2. The biological sensor according to claim 1,
wherein the second sheet includes a first conductive pattern disposed in a top portion of each of the plurality of projections.

3. The biological sensor according to claim 2,
wherein the second sheet includes a second conductive pattern disposed in a lateral surface of each of the plurality of projections and a portion of the second surface, in which no projection is formed, and
the second conductive pattern mutually connects a plurality of first conductive patterns each of which is the first conductive pattern.

4. The biological sensor according to claim 1, further comprising fluid disposed within the enclosed space in each of the plurality of projections.

5. The biological sensor according to claim 1,
wherein each of the plurality of projections has a meandering shape in plan view of the second sheet.

6. A method of manufacturing a biological sensor, the biological sensor comprising: a first sheet that has flexibility and/or stretchability; and a second sheet that has flexibility and/or stretchability, the second sheet having a first surface facing the first sheet, and a second surface opposite to the first surface, the method comprising:
forming a conductive pattern in the second surface of the second sheet;
forming in the second surface a plurality of projections that are configured to be brought into contact with a living body to obtain information on the living body; and
bonding a portion surrounding each of the plurality of projections in the first surface to the first sheet, to form, within each of the plurality of projections, an enclosed space defined by the first surface of the second sheet and the first sheet.
